(19) United States
(12) Patent Application Publication
(10) Pub. No.: US 2018/0088109 A1
(43) Pub. Date: Mar. 29, 2018

(54) BIOSENSOR STRIP

(71) Applicant: TELTRON CORPORATION, Daejeon (KR)

(72) Inventor: Jae Jin Lee, Daejeon (KR)

(21) Appl. No.: 15/332,584

(22) Filed: Oct. 24, 2016

(30) Foreign Application Priority Data
Sep. 29, 2016 (KR) 10-2016-0125182

(51) Int. Cl. G01N 33/543

(52) U.S. Cl. CPC G01N 33/54386

(57) ABSTRACT

A biosensor strip includes a housing composed of a hydrophobic material, a membrane formed on the housing and configured to move a liquid sample using a capillary phenomenon, a sample pad formed on one end of the membrane and including an inlet for feeding the liquid sample, a buffer layer formed under the sample pad and configured to uniformly distribute the liquid sample, an absorption pad formed on the remaining end of the membrane and configured to absorb a material that has moved through the membrane, and a reaction substrate formed on the membrane between the sample pad and the absorption pad and configured such that an antibody or aptamer is immobilized on an array of metal nanopatterns so as to react with an antigen of the liquid sample.

### BACKGROUND

### 1. Technical Field

The present techniques discussed in this application relate to a biosensor strip and, more particularly, to a biosensor strip, the analytical region of which is formed of a transparent material.

### 2. Description of the Related Art

A biosensor is a system configured such that information to be acquired from a measurement target may be efficiently converted into a recognizable signal, such as a color, fluorescence, an electrical signal, etc., using a biological factor or by simulating such a biological factor.

Recently useful as a biosensor is a biosensor strip based on an immuno-chromatographic assay using an immune response and an enzyme response.

Such a biosensor is configured such that the biosensor strip includes an opaque substrate and membrane, and thus changes on the surface thereof are observed with the naked eye or are analyzed based on light that is obliquely incident on and reflected from the structure. Hence, the fabrication of the biosensor analyzer is complicated, the volume thereof is large, and the measurement signal is weak due to the loss of light, making it difficult to perform analysis.

### SUMMARY

Accordingly, the present disclosure is intended to provide a biosensor strip, the analytical region of which is formed of a transparent material that enables the transmission of incident light, thereby improving the functionality of the analyzer and increasing resolution and the reproducibility of measurements.

Therefore, according to an embodiment, a biosensor strip is provided, including: a housing comprising a hydrophobic material, a membrane formed on the housing and configured to move a liquid sample using a capillary phenomenon, a sample pad formed on one end of the membrane and including an inlet for feeding the liquid sample, a buffer layer formed under the sample pad and configured to uniformly distribute the liquid sample, an absorption pad formed on a remaining end of the membrane and configured to absorb a material that has moved through the membrane, and a reaction substrate formed on the membrane between the sample pad and the absorption pad and configured such that an antibody or aptamer is immobilized on an array of metal nanopatterns so as to react with an antigen of the liquid sample.

The housing, the membrane, and the reaction substrate may comprise a material that enables transmission of incident light.

The housing may comprise any one material selected from among glass, a transparent polymer, and a transparent plastic.

The metal nanopatterns may be formed on a glass substrate, may have a size ranging from tens to hundreds of nm, and may be constituted using any one material selected from among gold, silver, aluminum, and copper.

The toxin concentration may be determined by transmitting incident light through the reaction substrate and measuring a wavelength shift of the transmitted light before and after an antigen-antibody reaction.

According to an embodiment, the analytical region of a biosensor strip is formed of a transparent material that enables the transmission of incident light, thereby increasing resolution and the reproducibility of measurements.

Also, according to an embodiment, a biosensor analyzer can be manufactured to be small.

### BRIEF DESCRIPTION OF THE DRAWINGS

The above and other features and advantages of the present techniques will be more clearly understood from the following detailed description taken in conjunction with the accompanying drawings, in which:

FIG. 1 is a cross-sectional view showing a biosensor strip according to an embodiment; and

FIG. 2 is a rear view of the reaction substrate of FIG. 1.

### DETAILED DESCRIPTION

Hereinafter, one or more embodiments will be described in detail with reference to the accompanying drawings so as to be easily performed by those skilled in the art. It should be appreciated that the embodiments may be implemented in various different forms, and are not limited to the embodiments described herein.

According to an embodiment, a biosensor strip is described below.

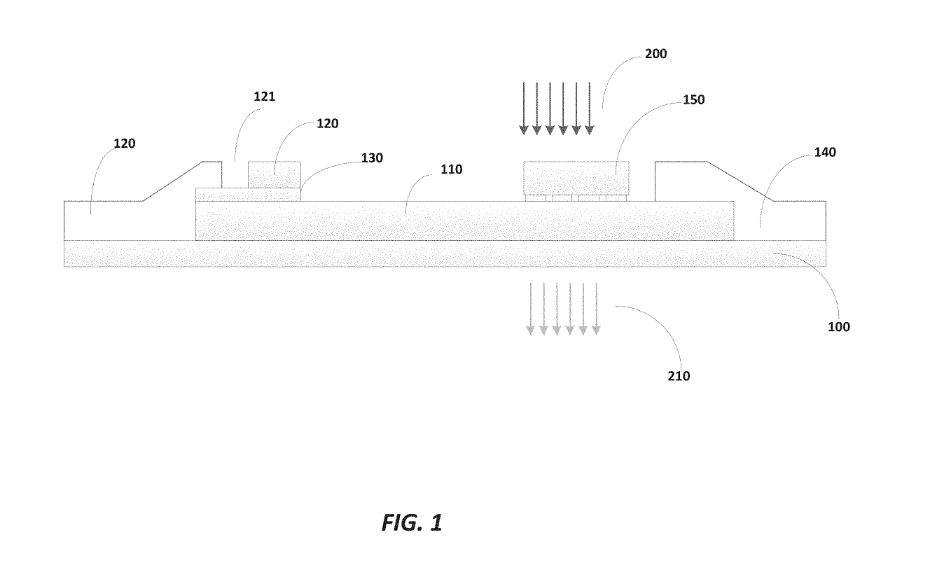

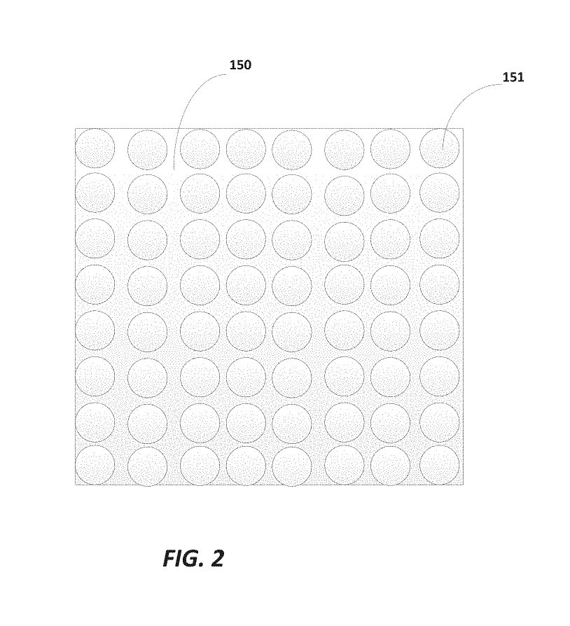

FIG. 1 is a cross-sectional view showing a biosensor strip according to an embodiment, and FIG. 2 is a rear view of the reaction substrate of FIG. 1.

As shown in FIGS. 1 and 2, the biosensor strip according to one example embodiment may be configured to include a housing 100, a membrane 110, a sample pad 120, a buffer layer 130, an absorption pad 140, and a reaction substrate 150.

The housing 100 is preferably made of a hydrophobic material, such as glass, a transparent polymer, a transparent plastic, etc.

The membrane 110 is provided on the housing 100 in the form of a transparent paper chip and functions to move a liquid sample using a capillary phenomenon. As such, when the liquid sample is moved using the capillary phenomenon, the membrane 110 is preferably treated with a protein, a polymer, a surfactant, etc. in order to prevent non-uniform flow and nonspecific reaction of the liquid sample.

The sample pad 120 is formed on one end of the membrane 110 and has an inlet 121 therein. The liquid sample, which is to be analyzed, is fed via the inlet 121.

The buffer layer 130 functions to uniformly distribute the liquid sample so as to be absorbed into the membrane 110. The buffer layer may be made of a material having pores the size of which is on the scale of ones of μm.

The absorption pad 140 is formed on the remaining end of the membrane 110 and is configured to absorb the material that has moved through the membrane 110. The absorption pad 140 absorbs surplus materials that are not bound by an immune response.

The reaction substrate 150 is formed on the membrane 110 between the sample pad 120 and the absorption pad 140. The reaction substrate 150 is configured such that metal nanopatterns 151 are provided in the form of an array on the glass substrate, and an antibody or aptamer is immobilized on the array of the metal nanopatterns 151 so as to react with the antigen of the liquid sample, whereby an antigen-antibody reaction occurs. The series of procedures for carrying out the antigen-antibody reaction can be any of various typical techniques that have been already known and performed prior to this application, and a detailed description thereof is omitted.

The metal nanopatterns 151 are formed to a size ranging from tens to hundreds of nm, and are constituted using any one material selected from among gold, silver, aluminum, and copper.

The housing 100, the membrane 11 and the reaction substrate 150 constitute the analytical region, and may be formed of a material that enables the transmission of incident light 200.

In the present embodiment, the toxin concentration of an analyte is measured by transmitting incident light 200 through the reaction substrate 150 on which the antigen-antibody reaction has occurred. Specifically, the transmitted light 210 may undergo a wavelength shift before and after the antigen-antibody reaction due to changes in refractive index depending on the reaction concentration. The toxin concentration of the analyte may be determined based on such a wavelength shift.

Although preferred embodiments have been disclosed for illustrative purposes, those skilled in the art will appreciate that various modifications, additions and substitutions are possible, without departing from the scope and spirit of the present disclosure as disclosed in the accompanying claims.

What is claimed is:

1. A biosensor strip, comprising:
a housing comprising a hydrophobic material;
a membrane formed on the housing and configured to move a liquid sample using a capillary phenomenon;
a sample pad formed on one end of the membrane and including an inlet for feeding the liquid sample;
a buffer layer formed under the sample pad and configured to uniformly distribute the liquid sample;
an absorption pad formed on a remaining end of the membrane and configured to absorb a material that has moved through the membrane; and
a reaction substrate formed on the membrane between the sample pad and the absorption pad and configured such that an antibody or aptamer is immobilized on an array of metal nanopatterns so as to react with an antigen of the liquid sample.

2. The biosensor strip of claim 1, wherein the housing, the membrane, and the reaction substrate comprise a material that enables transmission of incident light.

3. The biosensor strip of claim 1, wherein the housing comprises any one material selected from among glass, a transparent polymer, and a transparent plastic.

4. The biosensor strip of claim 1, wherein the metal nanopatterns are formed on a glass substrate, have a size ranging from tens to hundreds of nm, and are constituted using any one material selected from among gold, silver, aluminum, and copper.

5. The biosensor strip of claim 1, wherein a toxin concentration is determined by transmitting incident light through the reaction substrate and measuring a wavelength shift of the transmitted light before and after an antigen-antibody reaction.
